(19) United States
(12) Patent Application Publication
(10) Pub. No.: US 2023/0108048 A1
(43) Pub. Date: Apr. 6, 2023

(54) STANDARDIZED IMPLANT WITH INDIVIDUALIZED GUIDE TOOL

(71) Applicant: Episurf IP-Management AB, Stockholm (SE)

(72) Inventors: Katarina FLODSTRÖM, Danderyd (SE); Leif RYD, Malmö (SE)

(73) Assignee: Episurf IP-Management AB, Stockholm (SE)

(21) Appl. No.: 17/952,439

(22) Filed: Sep. 26, 2022

(30) Foreign Application Priority Data
Sep. 27, 2021 (SE) 2151182-9

(51) Int. Cl. A61B 34/10; A61F 2/46; G06T 7/00; G06T 17/00

(52) U.S. Cl. CPC A61B 34/10; A61F 2/4603; G06T 7/0012; G06T 17/00; A61B 2017/568

(57) ABSTRACT

A system 100 or designing a surgical kit 700 for articulating surface repair in an anatomical joint of a patient is provided, which comprises at least one processor 120 configured to: determine damage to the anatomical joint; select, from a predefined set of implants having varying dimensions, the implant 300 that is the best fit for repairing the determined damage; select, from a predefined set of insert tools, the insert tool 720 corresponding to the selected implant 300; and design a contact surface 540 of a guide tool 500 to have a shape and contour that is designed to correspond to and to fit the contour of a predetermined area. This enables the use of standardized implants 300 that can be manufactured batch-wise, while still using an individualized guide tool 500 to ensure correct positioning of the implant 300. This helps ensuring that the implant will be positioned in the exact location of the determined damage.

### TECHNICAL FIELD

The present disclosure relates generally to a surgical kit suitable for articulating surface repair in an anatomical joint of a patient.

### BACKGROUND

Pain and overuse disorders of the joints of the body is a common problem, that can be caused by for example injury, wear, arthritis, infection, or other cartilage and bone conditions. In a typical anatomical joint, the friction between the cartilage and the surrounding parts of the joint is very low, which facilitates movement of the joint under high pressure. The cartilage is however prone to damage due to disease, injury or chronic wear. Moreover, the cartilage does not readily heal after damages, as opposed to other connective tissue, and if healed, durable hyaline cartilage is often replaced by less durable fibrocartilage. This means that damages of the cartilage have a tendency to gradually become worse. It is therefore important to have efficient means and methods for repairing damaged cartilage and underlying bone, that may have developed into osteoarthritis, in joints.

The advantages of using implants for repairing damaged cartilage and underlying bone have stimulated the development of small joint implants, suitable for repair of injuries to cartilage and/or underlying bone that have a minimal influence on the surrounding parts of the joint. Such small implants are often designed with an implant body that may be formed as a plate with a wear resistant articulating surface for facing the articulate side of the joint, and a bone contacting surface for facing the bone below the damaged part of cartilage. The shape and the curvature of the articulating surface of the implant may be designed to be similar to the shape and the curvature of the part of the joint where the implant is inserted. Such small implants are often designed with a mushroom-like shape, having an implant body, or head, and one or more pegs, rods, or screws, projecting from the bone contacting side of the implant body for fastening the implant to the bone.

U.S. Pat. Nos. 8,655,468 and 8,644,973 describe the design of a surgical kit for cartilage repair in an anatomical joint of a patient. The surgical kit comprises an implant in which the curvature of the articulating surface is designed to correspond to the curvature of a simulated healthy surface at the site of diseased cartilage.

### Problems with the Prior Art

Individualized implants of the type described in U.S. Pat. Nos. 8,655,468 and 8,644,973 are relatively expensive to manufacture, since there can never be any type of batch-wise manufacturing of such implants. As implants typically require several production steps, such as machining, possibly coating, cleaning, and sterilization, the individualization of the implants adds substantially to the time and cost of manufacturing. This means that individualized implants may also lead to the patient having to wait very long for surgery.

Therefore, there is a need for an improved surgical kit for articulating surface repair in an anatomical joint of a patient.

### SUMMARY

The above described problem is addressed by the claimed system for designing a surgical kit for articulating surface repair in an anatomical joint of a patient. The system preferably comprises at least one processor configured to: obtain a three-dimensional image representation of the anatomical joint based on medical images generated using a medical imaging system; determine damage to the anatomical joint by analyzing medical images generated using a medical imaging system; select, from a predefined set of implants having varying dimensions, the implant that is the best fit for repairing the determined damage; select, from a predefined set of insert tools, each having an implant engaging portion that has a surface curvature that substantially corresponds to the surface curvature of the articulating surface of an implant in the predefined set of implants, the insert tool corresponding to the selected implant; and design a contact surface of a guide tool to have a shape and contour that is designed to correspond to and to fit the contour of the cartilage and/or the bone in a predetermined area that is located to allow the guide tool to guide a tool for creating an implant receiving surface that is shaped for receiving said selected implant at the site of the determined damage.

The above described problem is also addressed by the claimed method for designing a surgical kit for articulating surface repair in an anatomical joint of a patient. The method preferably comprises: obtaining a three-dimensional image representation of the anatomical joint based on medical images generated using a medical imaging system; determining damage to the anatomical joint by analyzing medical images generated using a medical imaging system; selecting, from a predefined set of implants having varying dimensions, the implant that is the best fit for repairing the determined damage; selecting, from a predefined set of insert tools, each having an implant engaging portion that has a surface curvature that substantially corresponds to the surface curvature of the articulating surface of an implant in the predefined set of implants, the insert tool corresponding to the selected implant; and designing a contact surface of a guide tool to have a shape and contour that is designed to correspond to and to fit the contour of the cartilage and/or the bone in a predetermined area that is located to allow the guide tool to guide a tool for creating an implant receiving surface that is shaped for receiving said selected implant at the site of the determined damage.

This enables the use of standardized implants that can be manufactured batch-wise, while still using an individualized guide tool to ensure correct positioning of the implant. This helps ensuring that the implant will be positioned in the exact location of the determined damage. Other dimensions of the guide tool, such as the diameter of a bore in the guide tool, are preferably designed to be adapted to the selected implant. There is preferably a matching insert tool for each standardized implant.

In embodiments, an implant dummy corresponding to the selected implant is selected from a predefined set of implant dummies having dimensions corresponding to the predefined set of implants. There is preferably a matching implant dummy for each standardized implant. If the implant receiving surface is created using a robot, there may be no need for an implant dummy, since a robot can be programmed to create a very exact implant receiving surface.

In embodiments, the selection of the implant involves simulating the surface curvature of the cartilage and/or the subchondral bone in a predetermined area comprising and surrounding the determined damage, and selecting the implant that has the best fit for the simulated surface curvature.

In embodiments, the contact surface of the guide tool is a cartilage contact surface, and the designing of the cartilage contact surface involves simulating the surface curvature of the cartilage and/or the subchondral bone in a predetermined area comprising and surrounding the determined damage.

The above described problem is also addressed by a non-transitory machine-readable medium on which is stored machine-readable code which, when executed by a processor, controls the processor to perform any one of the above described methods.

The above described problem is further addressed by the claimed surgical kit for articulating surface repair in an anatomical joint of a patient. The surgical kit preferably comprises: an implant selected from a predefined set of implants having varying dimensions; an insert tool corresponding to the selected implant, selected from a predefined set of insert tools, each having an implant engaging portion that has a surface curvature that substantially corresponds to the surface curvature of the articulating surface of an implant in the predefined set of implants, wherein the insert tool is configured to be used for pressing said implant to a suitably shaped implant receiving surface that has been e.g. drilled, milled, and/or sawed into the joint at the site of diseased cartilage and/or bone; and a guide tool comprising a contact surface configured to have a shape and contour that is designed to correspond to and to fit the contour of the cartilage and/or the bone in a predetermined area that is located to allow the guide tool to guide a tool for creating an implant receiving surface that is shaped for receiving said selected implant at a site of diseased cartilage and/or bone.

This enables the use of standardized implants that can be manufactured batch-wise, while still using an individualized guide tool to ensure correct positioning of the implant. This helps ensuring that the implant will be positioned in the exact location of the determined damage. There is preferably a matching insert tool for each standardized implant.

In embodiments, the surgical kit further comprises an implant dummy corresponding to the selected implant, selected from a predefined set of implant dummies having dimensions corresponding to the predefined set of implants. There is preferably a matching implant dummy for each standardized implant. If the implant receiving surface is created using a robot, there may be no need for an implant dummy, since a robot can be programmed to create a very exact implant receiving surface.

The above described problem is also addressed by the claimed method for inserting an implant comprising a positioning mark at a site of determined damage in an anatomical joint of a patient, for repairing damage in the anatomical joint. The method preferably comprises: attaching a guide tool to an articulating surface in the joint, the guide tool comprising a contact surface configured to have a shape and contour that is designed to correspond to and to fit the contour of the cartilage and/or the bone in a predetermined area that is located to allow the guide tool to guide a tool for creating an implant receiving surface that is shaped for receiving the implant at a site of diseased cartilage and/or bone; creating an implant receiving surface at the site of diseased cartilage and/or bone, e.g. by drilling, milling, and/or sawing the implant receiving surface, using the guide tool; removing the guide tool from the anatomical joint; rotating the implant so that the positioning mark on the implant is aligned with the marking on the cartilage; placing the implant on the implant receiving surface; and pressing the implant to the implant receiving surface, using an insert tool.

In embodiments, the method comprises making a marking on the cartilage at the side of the implant receiving surface, before removing the guide tool from the anatomical joint.

In embodiments, the method comprises verifying that the implant receiving surface fits the implant, using an implant dummy.

In embodiments, the method comprises applying an adhesive on the implant receiving surface, and/or on a bone contacting surface of the implant, before placing the implant on the implant receiving surface.

In embodiments, the implant receiving surface comprises a recess, into which at least a part of the implant, e.g. one or more implant pegs, is inserted.

In embodiments, the method is adapted to be at least partially automatically performed by a robot. In this case, there may be no need for an implant dummy, since a robot can be programmed to create a very exact implant receiving surface. It may be advantageous to use the robot only for creating the implant receiving surface, and manually place the implant on the implant receiving surface.

The articulating surface repair may repair damage to the cartilage and/or damage to the subchondral bone, since there may sometimes be lesions to the cartilage only, sometimes both in the cartilage and the subchondral bone, and sometimes in the subchondral bone even if there is no damage to the cartilage.

The medical imaging system may e.g. be a magnetic resonance imaging (MRI) system, an x-ray imaging system, an ultrasonic imaging system, a fluoroscopic imaging system and/or a computer tomography (CT) system, e.g. CBCT. The medical images may be a number of images in a series captured during a process of scanning through different layers of the anatomical joint or part of it using a medical imaging system.

The processor may in some embodiments comprise several different processors which together perform the claimed functions.

The scope of the invention is defined by the claims, which are incorporated into this section by reference. A more complete understanding of embodiments of the invention will be afforded to those skilled in the art, as well as a realization of additional advantages thereof, by a consideration of the following detailed description of one or more embodiments. Reference will be made to the appended sheets of drawings that will first be described briefly.

### BRIEF DESCRIPTION OF THE DRAWINGS

FIG. 1 shows a schematic view of a system for designing a surgical kit for articulating surface repair in an anatomical joint of a patient, in accordance with one or more embodiments described herein.

FIGS. 2a-2b show two in-lay implants, in accordance with one or more embodiments described herein, in a model of the knee joint.

FIGS. 3a-3b illustrate a guide tool for a knee trochlear implant, in accordance with one or more embodiments described herein.

FIGS. 3c-3d illustrate a guide tool for a patellar implant, in accordance with one or more embodiments described herein.

FIG. 4 illustrates a guide tool for an elongated (twin) implant, in accordance with one or more embodiments described herein.

FIGS. 5a-5d illustrate embodiments of a guide tool for a metatarsal implant, in accordance with one or more embodiments described herein.

FIGS. 6a-6d illustrate the creation of an implant receiving surface for an implant, in accordance with one or more embodiments described herein.

FIG. 7 illustrates a surgical kit, in accordance with one or more embodiments described herein.

FIG. 8 is a schematic flow diagram for a method for customizing an implant, in accordance with one or more embodiments described herein.

FIG. 9 is a schematic flow diagram for a method for inserting an implant at a site of determined damage in an anatomical joint of a patient, in accordance with one or more embodiments described herein.

Embodiments of the present disclosure and their advantages are best understood by referring to the detailed description that follows. It should be appreciated that like reference numerals are used to identify like elements illustrated in one or more of the figures.

### DETAILED DESCRIPTION

### Introduction

Individualized implants are relatively expensive to manufacture, since there can never be any type of batch-wise manufacturing of such implants. As implants typically require several production steps, such as machining, possibly coating, cleaning, and sterilization, the individualization of the implants adds substantially to the cost of manufacturing. If standardized implants are used instead of individualized implants, this enables the batch-wise manufacturing of a set of standardized implants of different dimensions that may be stored to be later used for repairing damage in an anatomical joint. Especially for anatomical joints for which there is not a large anatomical variation between patients, this is advantageous. This enables more efficient production processes, while still using an individualized guide tool to ensure correct positioning of the implant. This helps ensuring that the implant will be positioned in the exact location of the determined damage.

The present disclosure relates generally to a surgical kit for articulating surface repair in an anatomical joint of a patient. The articulating surface repair may repair damage to the cartilage and/or damage to the subchondral bone, since there may sometimes be lesions to the cartilage only, sometimes both in the cartilage and the subchondral bone, and sometimes in the subchondral bone even if there is no damage to the cartilage. Embodiments of the disclosed solution are presented in more detail in connection with the figures.

System Architecture

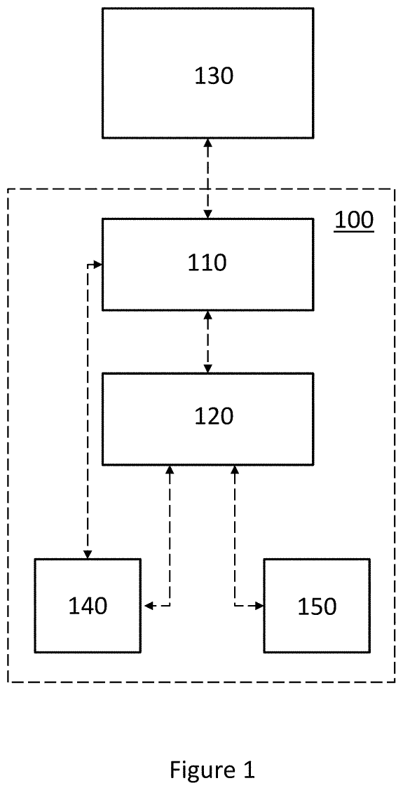

FIG. 1 shows a schematic view of a system 100 for designing a surgical kit for articulating surface repair in an anatomical joint of a patient. The implant may be configured to be inserted, preferably with press-fit, into a recess in the anatomical joint in such a way that the perimeter of an articulating surface of the implant does not extend beyond the surrounding articulating surface. Other types of implants are instead placed on an implant receiving surface on the anatomical joint; such an implant receiving surface may however also comprise a recess, for receiving a part of the implant. According to embodiments, the system 100 comprises a display 140, at least one manipulation tool 150, and a storage media 110, configured to receive and store image data and parameters. In some embodiments, the system 100 is communicatively coupled to a medical imaging system 130. The medical imaging system 130 may be configured to capture or generate medical images, e.g. radiology images such as X-ray images, ultrasound images, computed tomography (CT), e.g. CBCT, images, nuclear medicine including positron emission tomography (PET) images, and magnetic resonance imaging (MRI) images. The storage media 110 may be configured to receive and store medical images from the medical imaging system 130. In embodiments, medical images are uploaded into the storage media 110 by personnel at a medical care facility, preferably the medical care facility where the medical imaging takes place. Medical images may however also be uploaded into the storage media 110 by another medical care facility, or by other authorized personnel. The uploading of the medical images may also be an automatic uploading directly from one system to another.

In one or more embodiments, the system 100 comprises at least one processor 120 configured to: obtain a three-dimensional image representation of the anatomical joint based on medical images generated using a medical imaging system 130; determine damage to the anatomical joint by analyzing medical images generated using a medical imaging system 130; select, from a predefined set of implants having varying dimensions, the implant that is the best fit for repairing the determined damage; select, from a predefined set of insert tools, each having an implant engaging portion that has a surface curvature that substantially corresponds to the surface curvature of the articulating surface 310 of an implant in the predefined set of implants, the insert tool 720 corresponding to the selected implant 300; and design a contact surface of a guide tool to have a shape and contour that is designed to correspond to and to fit the contour of the cartilage and/or the bone in a predetermined area that is located to allow the guide tool to guide a tool for creating an implant receiving surface that is shaped for receiving said selected implant at the site of the determined damage. There is preferably a matching insert tool for each standardized implant.

In one or more embodiments, the at least one processor 120 is configured to select, from a predefined set of implant dummies having dimensions corresponding to the predefined set of implants, the implant dummy corresponding to the selected implant 300. There is preferably a matching implant dummy for each standardized implant. If the implant receiving surface is created using a robot, there may be no need for an implant dummy, since a robot can be programmed to create a very exact implant receiving surface.

In one or more embodiments, the at least one processor 120 is configured to select the implant as the best fit for a simulated surface curvature of the cartilage and/or the subchondral bone in a predetermined area comprising and surrounding the determined damage. Since the selection of the implant is made by the at least one processor 120, what is selected is an electronic representation having the same dimensions as the physical implant.

In one or more embodiments, the at least one processor 120 is configured to design the contact surface of the guide tool as a cartilage contact surface based on a simulated surface curvature of the cartilage and/or the subchondral bone in a predetermined area comprising and surrounding the determined damage.

It is desirable to simulate the curvature of the cartilage surface as closely as possible. If just the 3D curvature of the subchondral bone subjacent to an area of damaged cartilage is used for simulating the cartilage surface, for selecting an implant as the best fit for the cartilage surface curvature, this does not necessarily correspond to the cartilage surface, since the cartilage does not necessarily have uniform thickness. However, for implants in anatomical parts where the cartilage does have a substantially uniform thickness, the 3D curvature of the subchondral bone subjacent to the area of damaged cartilage may be used for selecting the implant.

According to embodiments, the healthy surface is instead simulated based on the curvature of the cartilage surrounding the area of damaged cartilage. Preferably, a suitable area comprising and extending around the damaged cartilage is selected, and the curvature of the whole area is simulated in such a way that the curvature of the area which is not damaged matches the actual curvature, and a simulated healthy surface of the area of damaged cartilage is generated. The simulation may comprise an interpolation, e.g. using the Solid Works Surface Wizard or another suitable tool. The determined damage may be marked as more or less severe. Damage that is very mild may not need repairing, but severe lesions should preferably have a surface coverage of at least 90%.

The at least one processor 120 may for example be a general data processor, or other circuit or integrated circuit capable of executing instructions to perform various processing operations. The at least one processor 120 may in some embodiments comprise several different processors 120 which together perform the claimed functions. In the same way, the storage media 110 may in some embodiments comprise several different storage media 110 which together perform the claimed functions.

The display 140 may be configured to receive image data for display via the processor 120, and/or to retrieve image data for display directly from the storage media 110, possibly in response to a control signal received from the processor 120 or the at least one manipulation tool 150.

The processor 120 may further be configured to perform any or all of the method steps of any or all of the embodiments presented herein.

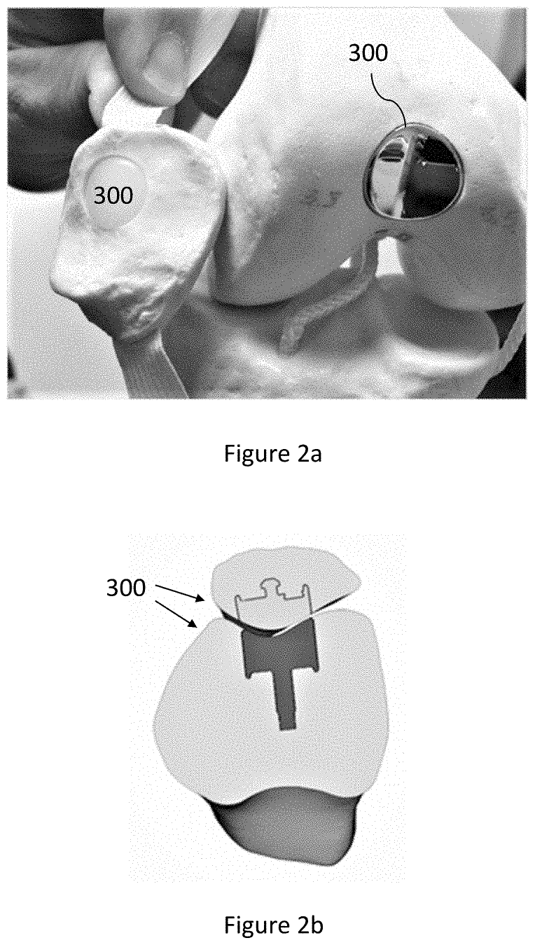

FIGS. 2a-b shows two in-lay implants 300 in a model of the knee joint. The implants 300 comprise articulating surfaces 310 that are designed to allow that they at least partly interact with each other when the implants 300 are implanted into the anatomical joint. Each implant 300 is configured to be inserted, preferably with press-fit, into a recess in the anatomical joint in such a way that the perimeter of the articulating surface 310 of the implant 300 does not extend beyond a surrounding articulating surface. The definition that the implant 300 “does not extend beyond” a surrounding articulating surface also covers if the perimeter of the articulating surface 310 of the implant 300 after insertion is approximately level with the surrounding articulating surface. In practice, it is however often desired that the perimeter of the articulating surface 310 of the implant 300 is slightly (e.g. 0,5-1 mm) lower than the surrounding articulating surface. This ensures that there will be no sharp implant edges sticking out of the surrounding cartilage, even if the surrounding cartilage is compressed when loaded; such sharp edges may be painful for the patient. This also enables a thin layer of tissue to grow on the articulating surface 310 of the implant 300.

The articulating surface 310 of one of the implants 300 is preferably a metal or ceramic surface, e.g. comprising titanium (Ti) or titanium alloy, titanium nitride (TiN), titanium niobium nitride (TiNbN), and/or a cobalt-chromium (CoCr) alloy. In order to avoid a very hard surface, such as a metal or ceramic surface, interfacing with another very hard surface, creating e.g. a metal-on-metal interface, the other implant 300 preferably has an articulating surface 310 that is not a metal or ceramic surface. The articulating surface 310 of the other implant 300 is preferably a polymer surface, e.g. a surface of polyethylene, e.g. the polyethylene UHMWPE (e.g. cross-linked UHMWPE or vitamin E enhanced UHMWPE). Preferably, the whole implant 300 is manufactured from the same polymer material, since this simplifies the manufacturing process. The main body of the implant 300 may however be manufactured from metal or ceramic, if the articulating surface 310 comprises a polymer material, such as polyethylene, e.g. the polyethylene UHMWPE. If the bone contacting surface of the implant 300 is a non-porous metal or ceramic surface, it may be advantageous to coat the bone contacting surface with an osseointegrating and/or bioactive material, such as e.g. hydroxyapatite. This reduces the need for using an adhesive for to securing the implant 300 to the implant receiving surface on the anatomical joint.

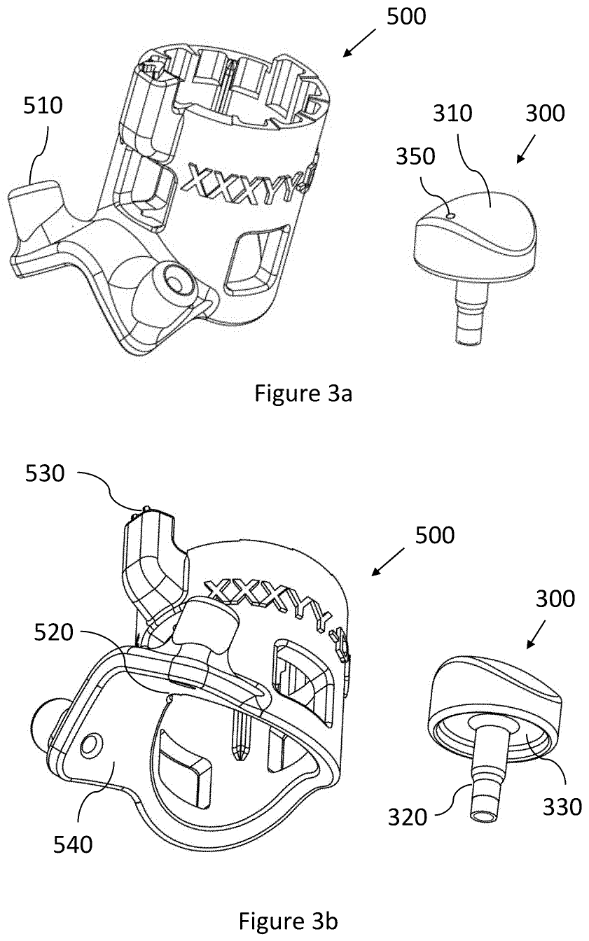

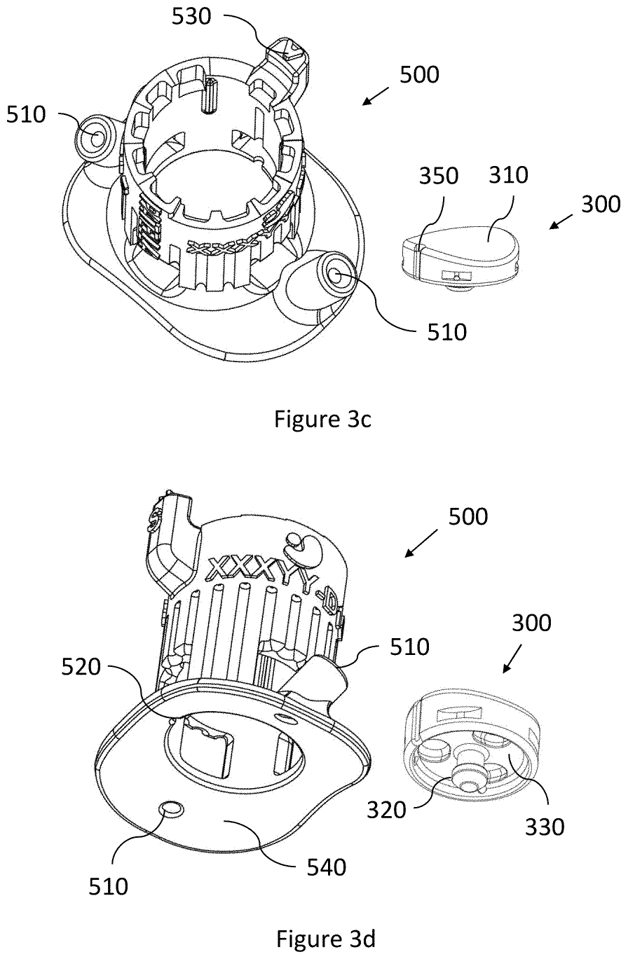

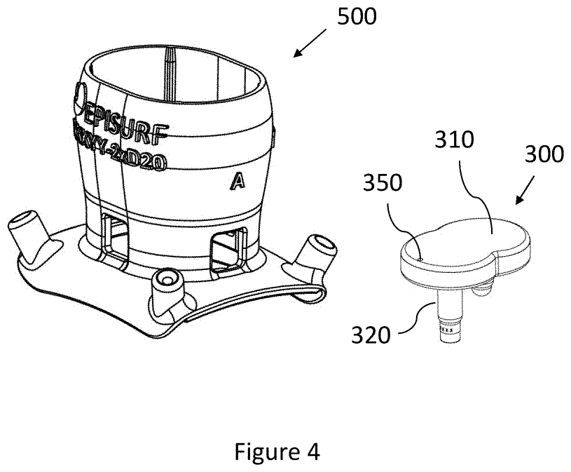

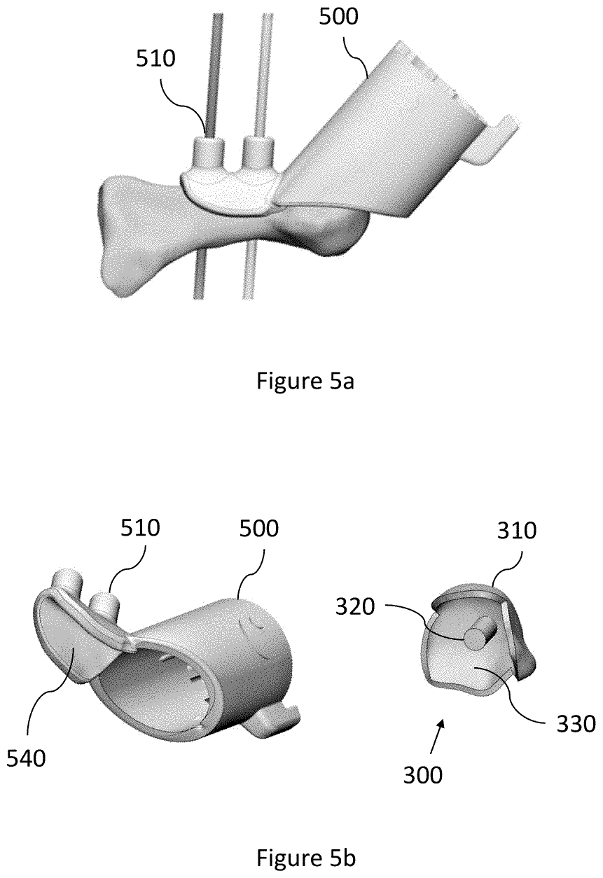

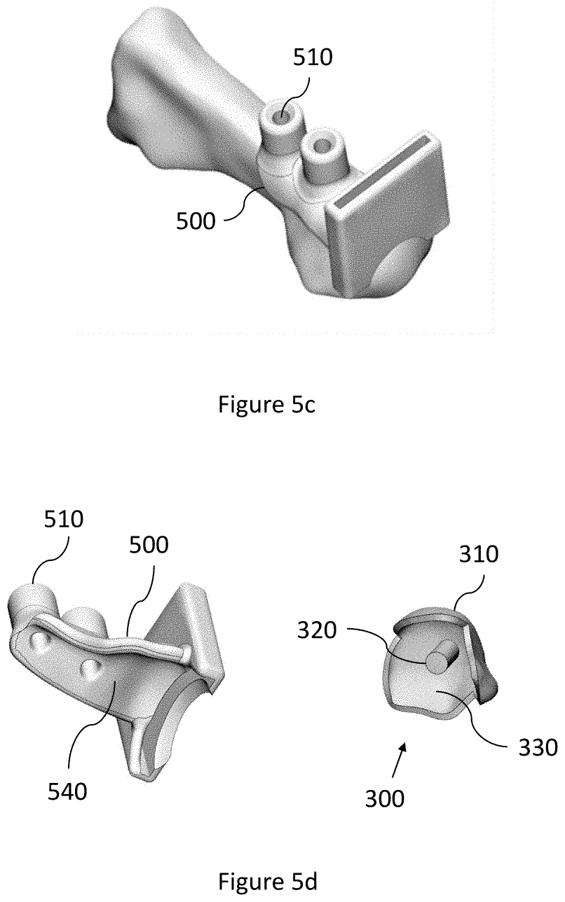

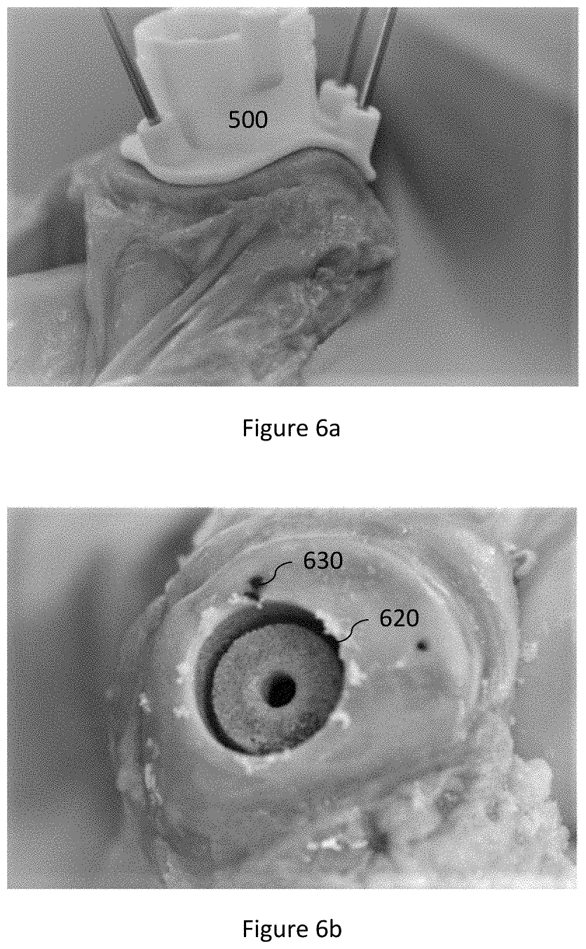

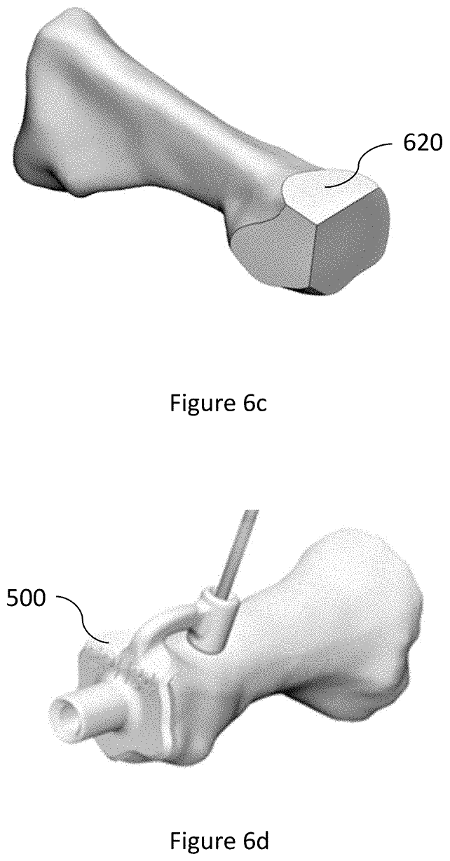

FIGS. 3a-b illustrate a guide tool 500 for a knee trochlear implant 300, FIGS. 3c-d illustrate a guide tool 500 for a patellar implant 300, FIG. 4 illustrates a guide tool 500 for an elongated (twin) implant 300, FIGS. 5a-d illustrate embodiments of a guide tool 500 for a metatarsal implant, and FIGS. 6a-d illustrate the creation of an implant receiving surface 620 for an implant 300, where a guide tool 500 is attached to an anatomical joint and used for guiding the tool for drilling, milling, and/or sawing. FIGS. 6a-b illustrate the drilling of a recess 620 for an implant 300, FIG. 6c illustrates an implant receiving surface 620 that has been sawed and does therefore not yet comprise a recess, and FIG. 6d illustrates a drill guide 600 that has a contact surface that is adapted to fit the sawed implant receiving surface 620. The drill guide 600 is preferably used to guide a drill for drilling a recess for the implant peg 320 on the implant receiving surface 620 of FIG. 6c.

As illustrated, the implant 300 may have different shapes. Knee trochlear implants 300 and patellar implants 300 preferably have an approximately cylindrical shape, or a shape that corresponds to a plurality of partly overlapping cylinders. Metatarsal implants 300 are preferably shaped to enclose the distal end of the metatarsal bone (the metatarsal head). Regardless of the shape, the implant 300 preferably comprises one or more implant pegs 320 extending from a suitably shaped bone contacting surface 330. Before the implant 300 is placed on a suitably shaped implant receiving surface 620, preferably comprising a recess for the implant peg 320, in the anatomical joint, the implant receiving surface 620 may be filled with an adhesive such as e.g. bone cement. Bone cement consists of powder, most commonly polymethylmethacrylate (PMMA), mixed with liquid. If an adhesive such as bone cement is used, it may not be necessary for the implant 300 to be designed for press-fit in the recess of the implant receiving surface 620. The use of press-fit (where the part of the implant that is pressed into the recess is slightly larger than the recess) secures the implant 300 to the implant receiving surface 620 regardless of whether an adhesive such as bone cement is used, but the combination of press-fit and adhesive of course secures the implant 300 even more to the implant receiving surface 620.

The bone contacting surface 330 of the implant 300 may be coated with an osseointegrating and/or bioactive material, such as e.g. hydroxyapatite. In such a case, it may not be necessary to use an adhesive. It may however be difficult to coat a polymer material with an osseointegrating and/or bioactive material, so the use of an osseointegrating and/or bioactive coating on the bone contacting surface is simplified if the body of the implant 300 is manufactured from metal or ceramic, even though the articulating surface 310 comprises a polymer material.

The implant 300 may also comprise a positioning mark 350, preferably positioned on the articulating surface 310. The positioning mark 350 may in embodiments extend also to the side of the implant 300, e.g. in the form of an indentation 350 in the side of the implant 300, as illustrated in FIGS. 3c-d.

The surface curvature of the articulating surface 310 of the implant 300 preferably corresponds as closely as possible to the surface curvature of the original, undamaged, anatomical joint. By analyzing the surface curvature of the cartilage and/or the subchondral bone in a predetermined area comprising and surrounding the site of diseased cartilage, it is possible to simulate a healthy articulating surface and mimic the original, undamaged, articulating surface. The image data may be analyzed in a data processing system to identify and determine physical parameters for the cartilage damage. The physical parameters to be determined may comprise the presence, the location and the size and shape of the cartilage damage, as well as curvature of the surface contour of the cartilage or the subchondral bone in an area of the cartilage damage.

When such a healthy articulating surface has been simulated, it is possible to select the best matching predefined surface from a limited number of different predefined surfaces. This enables the use of standardized implants 300. In this way, a set of standardized implants 300 of different dimensions may be manufactured and stored, to be later used for repairing damage in the anatomical joint. This is advantageous because the production process for entirely individualized implants 300 is normally much less efficient than the batch-wise manufacturing of implants 300, especially for implants 300 that are not manufactured from metal.

A standardized implant 300 may in this case be selected from a predefined set of standardized implants 300 having varying dimensions. The dimensions may vary e.g. regarding shape (e.g. cylindrical or a shape that corresponds to a plurality of partly overlapping cylinders) and diameters for the one or more cylinders, in addition to the variation in the surface curvatures of the standardized implants 300. The predefined set of standardized implants 300 is preferably created by analyzing dimensional data from stored images of the relevant anatomical joint from a large number of different patients. The standardized implant 300 should be selected as a standardized implant 300 having dimensions that match a determined damage, thereby making it suitable for repairing the determined damage. A 3D model of the anatomical joint, visualizing the determined damage, may be used in order to determine which standardized implant 300 is the best fit for repairing the determined damage. What is selected is then an electronic representation having the same dimensions as the physical implant 300.

The guide tool 500 preferably has a contact surface 540 that has a shape and contour that is designed to correspond to and to fit the contour of the articulating surface of the anatomical joint in a predetermined area at the site of diseased cartilage. Thereby, the contact surface 540 of the guide tool 500 corresponds to and fits to the surface contour of the articulating surface. The whole contact surface 540 does not have to correspond to the contour curvature of the articulating surface of the anatomical joint, it is enough if the contact surface 540 comprises at least three cartilage contact points, so that the guide tool 500 will have a stable mounting in the correct position in the anatomical joint. Such cartilage contact points are preferably chosen to provide maximum support and positional stability for the guide tool 500. This helps ensuring that the implant receiving surface 620 will be created in the exact position of the determined damage.

The contact surface 540 of the guide tool 500 may be further stabilized by being attached to the anatomical joint with one or more nails, rivets, wires or similar attachment means, as illustrated in FIGS. 5a and 6a. Such additional attachment gives additional support and stability, and enables the contact surface 540 of the guide tool 500 to be as small as possible. The guide tool 500 may comprise through-holes 510, through which the one or more nails, rivets, wires or similar attachment means may be inserted, in order to attach the guide tool 500 to the anatomical joint.

The guide tool 500 may comprise markings to help the surgeon understand how it should be placed, such as e.g. “A” for “anterior” and “P” for “posterior”. The surgeon also preferably has access to images that show how the guide too 500 should be placed in the anatomical joint.

The guide tool 500 may comprise a rotational position indicator 530, which may be used to make a marking 630 on the cartilage at the side of the implant receiving surface 620, as illustrated in FIG. 6b. Such a marking 630 may then be used to correctly rotate the implant 300 when the implant 300 is placed on the implant receiving surface 620. If the implant 300 comprises a positioning mark 350, the alignment of this positioning mark 350 with the marking 630 on the cartilage at the side of the implant receiving surface 620 ensures that the implant 300 is correctly rotated on the implant receiving surface 620. The guide tool 500 is preferably configured to allow such a marking 630 to be made while the guide tool 500 is attached to the anatomical joint. The guide tool 500 may for this purpose comprise an indentation 520 at the position of the rotational position indicator 530 on the guide tool 500. The marking 630 may e.g. be added to the cartilage surface by inserting a marking pen into the indentation 520 in the guide tool 500 when the guide tool 500 is attached to the anatomical joint.

A correct rotational positioning of the implant 300 is important because the articulating surface 310 of the implant 300 will in most situations not be rotationally symmetric. An important reason for selecting the articulating surface 310 of the implant 300 as the best fit to the simulated healthy articulating surface of the anatomical joint is to ensure that the implant 300 fits smoothly on the implant receiving surface 620, with no sharp edges. If the implant 300 is not mounted with a correct rotational positioning, there may be sharp edges even though the articulating surface 310 of the implant 300 has been selected as the best fit to the simulated healthy articulating surface of the anatomical joint. Such sharp edges may be painful for the patient.

An implant dummy 710 having dimensions corresponding to the implant 300 may be used for verifying that the drill depth is correct before the guide tool 500 is removed. When the predefined set of standardized implants having varying dimensions is created, a predefined set of matching implant dummies is preferably created as well. The selection of an implant 300 in this way preferably automatically leads to the selection of a matching implant dummy 710. If the implant receiving surface is created using a robot, there may be no need for an implant dummy 710, since a robot can be programmed to create a very exact implant receiving surface.

The implant 300 is preferably attached to the implant receiving surface 620 using an adhesive, e.g. bone cement, that is applied on the implant receiving surface 620 and/or on the bone contacting surface 330 of the implant 300, before the implant 300 is placed on the implant receiving surface 620.

One or more insert tools 720 may be used to aid the pressing of the implant to the implant receiving surface 620 in the anatomical joint. It is e.g. possible to use a mandrel as an insert tool, as is commonly known for knee trochlear implants, especially if the implant 300 is designed with press-fit into the recess of the implant receiving surface 620. The insert tool 720 preferably comprises an implant engaging portion, which preferably has a surface curvature that substantially corresponds to the surface curvature of the articulating surface 310 of the implant 300. When the predefined set of standardized implants having varying dimensions is created, a predefined set of matching insert tools is preferably created as well. The selection of an implant 300 in this way preferably automatically leads to the selection of a matching insert tool 720.

A marking 630 on the cartilage surface makes it easy for the surgeon to place the implant 300 with a correct rotational positioning, if the implant 300 also comprises a positioning mark 350. Preferably, there is a positioning mark also on the insert tool 720, so that the implant engaging portion may be correctly rotated with respect to the implant 300.

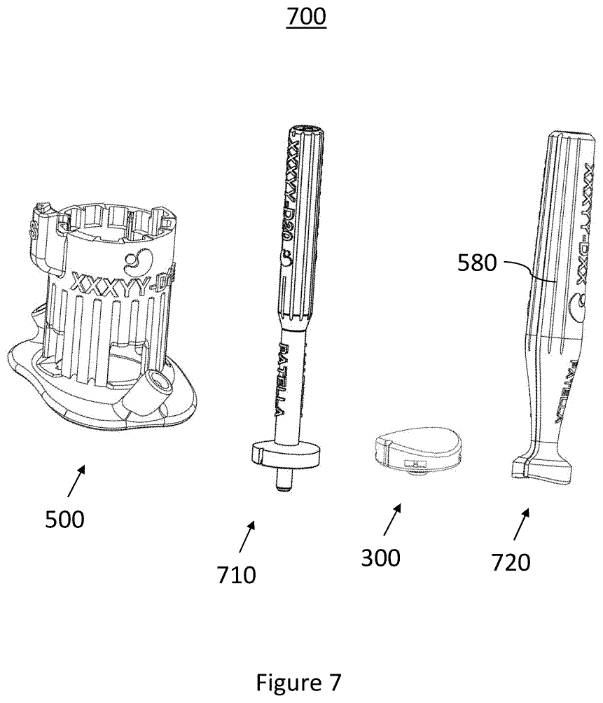

In order to repair the damaged cartilage in the anatomical joint, a surgical kit 700 comprising the above described implant 300, the above described guide tool 500, and the above described insert tool 720 may be used. Even though the implant 300 is an implant selected from a predefined set of standardized implants having varying dimensions, it is still preferred to use a customized guide tool 500, having a contact surface 540 configured to have a shape and contour that is designed to correspond to and to fit the contour of the cartilage and/or the bone in a predetermined area that is located to allow the guide tool 500 to guide a tool for creating an implant receiving surface 620 that is shaped for receiving the selected implant 300 at a site of diseased cartilage, since this will ensure that the guide tool 500 will have a stable mounting in the correct position in the anatomical joint. This helps ensuring that the implant receiving surface 620 will be created in the exact position of the determined damage. A surgical kit 700 may also comprise further instruments, such as e.g. the above described implant dummy 710, one or more inserts into the guide tool 500, to enable precision in the drilling process, and/or a drill bit, possibly adapted to the specific implant 300. If the implant receiving surface is created using a robot, there may be no need for an implant dummy 710, since a robot can be programmed to create a very exact implant receiving surface. FIG. 7 illustrates a surgical kit 700 comprising a guide tool 500, an implant dummy 710, an implant 300, and an insert tool 720 in the form of a mandrel to aid the pressing of the implant 300 to the implant receiving surface 620.

### METHOD EMBODIMENTS

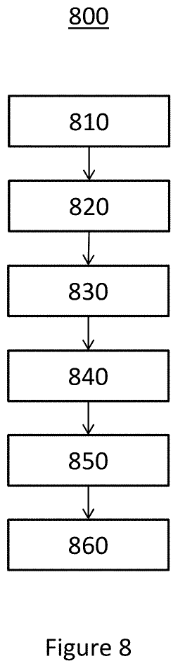

FIG. 8 is a flow diagram of embodiments of a method 800 for designing a surgical kit 700 for articulating surface repair in an anatomical joint of a patient. In accordance with one or more embodiments, the method 800 comprises:

Step 810: obtaining a three-dimensional image representation of the anatomical joint based on medical images generated using a medical imaging system 130.

Step 820: determining damage to the anatomical joint by analyzing medical images generated using a medical imaging system 130.

Step 830: selecting, from a predefined set of implants having varying dimensions, the implant 300 that is the best fit for repairing the determined damage.

Step 850: selecting, from a predefined set of insert tools, each having an implant engaging portion that has a surface curvature that substantially corresponds to the surface curvature of the articulating surface 310 of an implant in the predefined set of implants, the insert tool 720 corresponding to the selected implant 300.

Step 860: designing a contact surface 540 of a guide tool 500 to have a shape and contour that is designed to correspond to and to fit the contour of the cartilage and/or the bone in a predetermined area that is located to allow the guide tool 500 to guide a tool for creating an implant receiving surface 620 that is shaped for receiving said selected implant 300 at the site of the determined damage.

This enables the use of standardized implants that can be manufactured batch-wise, while still using an individualized guide tool 500 to ensure correct positioning of the implant 300. This helps ensuring that the implant 300 will be positioned in the exact location of the determined damage. There is preferably a matching insert tool 720 for each standardized implant 300.

In embodiments, the selection 830 of the implant 300 involves simulating the surface curvature of the cartilage and/or the subchondral bone in a predetermined area comprising and surrounding the determined damage, and selecting the implant that has the best fit for the simulated surface curvature.

In embodiments, the contact surface 540 of the guide tool 500 is a cartilage contact surface, and the designing 840 of the cartilage contact surface 540 involves simulating the surface curvature of the cartilage and/or the subchondral bone in a predetermined area comprising and surrounding the determined damage.

The method 800 may further comprise:

Step 840: selecting, from a predefined set of implant dummies having dimensions corresponding to the predefined set of implants, the implant dummy 710 corresponding to the selected implant 300. There is preferably a matching implant dummy 710 for each standardized implant 300.

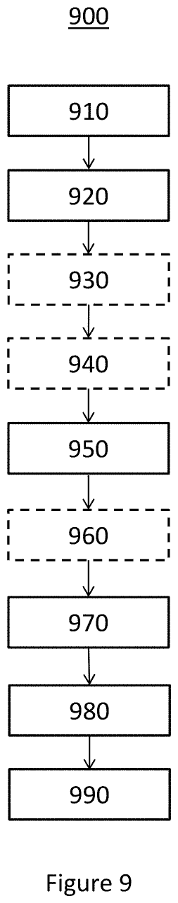

FIG. 9 is a flow diagram of embodiments of a method 900 for inserting an implant 300 comprising a positioning mark 350 at a site of determined damage in an anatomical joint of a patient, for repairing damage in the anatomical joint. In accordance with one or more embodiments, the method 900 comprises:

Step 910: attaching a guide tool 500 to the articulating surface in the joint, the guide tool 500 comprising a contact surface 540 configured to have a shape and contour that is designed to correspond to and to fit the contour of the cartilage and/or the bone in a predetermined area that is located to allow the guide tool 500 to guide a tool for creating an implant receiving surface 620 that is shaped for receiving the implant 300 at a site of diseased cartilage and/or bone.

Step 920: creating an implant receiving surface 620 at the site of diseased cartilage and/or bone, e.g. by drilling, milling, and/or sawing the implant receiving surface 620, using the guide tool 500.

Step 950: removing the guide tool 500 from the anatomical joint.

Step 970: rotating the implant 300 so that the positioning mark 350 on the implant 300 is aligned with the marking 630 on the cartilage. This may take place simultaneously with step 980.

Step 980: placing the implant 300 on the implant receiving surface 620.

Step 990: pressing the implant 300 to the implant receiving surface 620, using an insert tool 720.

In embodiments, the method 900 also comprises one or more of: Step 930: making a marking 630 on the cartilage at the side of the implant receiving surface 620.

Step 940: verifying that the implant receiving surface 620 fits the implant 300, using an implant dummy 710.

Step 960: applying an adhesive on the implant receiving surface 620 and/or on a bone contacting surface 330 of the implant 300 before placing the implant 300 on the implant receiving surface 620.

In embodiments, the implant receiving surface 620 comprises a recess, into which at least a part of the implant 300, such as e.g. one or more implant pegs 320, is inserted.

In embodiments, the method is adapted to be at least partially automatically performed by a robot. In this case, there may be no need for an implant dummy 710, since a robot can be programmed to create a very exact implant receiving surface.

### Further Embodiments

Where applicable, various embodiments provided by the present disclosure can be implemented using hardware, software, or combinations of hardware and software. Also where applicable, the various hardware components and/or software components set forth herein can be combined into composite components comprising software, hardware, and/or both without departing from the claimed scope of the present disclosure. Where applicable, the various hardware components and/or software components set forth herein can be separated into sub-components comprising software, hardware, or both without departing from the claimed scope of the present disclosure. In addition, where applicable, it is contemplated that software components can be implemented as hardware components, and vice-versa. The method steps of one or more embodiments described herein may be performed automatically, by any suitable processing unit, or one or more steps may be performed manually. Where applicable, the ordering of various steps described herein can be changed, combined into composite steps, and/or separated into sub-steps to provide features described herein.

Software in accordance with the present disclosure, such as program code and/or data, can be stored in non-transitory form on one or more machine-readable mediums. It is also contemplated that software identified herein can be implemented using one or more general purpose or specific purpose computers and/or computer systems, networked and/or otherwise.

In embodiments, there are provided a computer program product comprising computer readable code configured to, when executed in a processor, perform any or all of the method steps described herein. In some embodiments, there are provided a non-transitory computer readable memory on which is stored computer readable and computer executable code configured to, when executed in a processor, perform any or all of the method steps described herein.

In one or more embodiments, there is provided a non-transitory machine-readable medium on which is stored machine-readable code which, when executed by a processor, controls the processor to perform the method of any or all of the method embodiments presented herein.

The foregoing disclosure is not intended to limit the present invention to the precise forms or particular fields of use disclosed. It is contemplated that various alternate embodiments and/or modifications to the present invention, whether explicitly described or implied herein, are possible in light of the disclosure. Accordingly, the scope of the invention is defined only by the claims.

1. System for designing a surgical kit for articulating surface repair in an anatomical joint of a patient, the system comprising at least one processor configured to:
obtain a three-dimensional image representation of the anatomical joint based on medical images generated using a medical imaging system;
determine damage to the anatomical joint by analyzing medical images generated using a medical imaging system;
select, from a predefined set of implants having varying dimensions, the implant that is the best fit for repairing the determined damage;
select, from a predefined set of insert tools, each having an implant engaging portion that has a surface curvature that substantially corresponds to the surface curvature of the articulating surface of an implant in the predefined set of implants, the insert tool corresponding to the selected implant; and
design a contact surface of a guide tool to have a shape and contour that is designed to correspond to and to fit the contour of the cartilage and/or the bone in a predetermined area that is located to allow the guide tool to guide a tool for creating an implant receiving surface that is shaped for receiving said selected implant at the site of the determined damage.

2. System according to claim 1, wherein the at least one processor is further configured to select, from a predefined set of implant dummies having dimensions corresponding to the predefined set of implants, the implant dummy corresponding to the selected implant.

3. System according to claim 1, wherein the at least one processor is configured to select the implant as the best fit for a simulated surface curvature of the cartilage and/or the subchondral bone in a predetermined area comprising and surrounding the determined damage.

4. System according to claim 1, wherein the at least one processor is configured to design the contact surface of the guide tool as a cartilage contact surface based on a simulated surface curvature of the cartilage and/or the subchondral bone in a predetermined area comprising and surrounding the determined damage.

5. Method for designing a surgical kit for articulating surface repair in an anatomical joint of a patient, the method comprising:
obtaining a three-dimensional image representation of the anatomical joint based on medical images generated using a medical imaging system;
determining damage to the anatomical joint by analyzing medical images generated using a medical imaging system;
selecting, from a predefined set of implants having varying dimensions, the implant that is the best fit for repairing the determined damage;
selecting, from a predefined set of insert tools, each having an implant engaging portion that has a surface curvature that substantially corresponds to the surface curvature of the articulating surface of an implant in the predefined set of implants, the insert tool corresponding to the selected implant; and
designing a contact surface of a guide tool to have a shape and contour that is designed to correspond to and to fit the contour of the cartilage and/or the bone in a predetermined area that is located to allow the guide tool to guide a tool for creating an implant receiving surface that is shaped for receiving said selected implant at the site of the determined damage.

6. Method according to claim 5, further comprising selecting, from a predefined set of implant dummies having dimensions corresponding to the predefined set of implants, the implant dummy corresponding to the selected implant.

7. Method according to claim 5, wherein the selection of the implant involves simulating the surface curvature of the cartilage and/or the subchondral bone in a predetermined area comprising and surrounding the determined damage, and selecting the implant that has the best fit for the simulated surface curvature.

8. Method according to claim 5, wherein the contact surface of the guide tool is a cartilage contact surface, and the designing of the cartilage contact surface involves simulating the surface curvature of the cartilage and/or the subchondral bone in a predetermined area comprising and surrounding the determined damage.

9. Non-transitory machine-readable medium on which is stored machine-readable code which, when executed by at least one processor, controls the processor to perform the method of claim 5.

10. Surgical kit for articulating surface repair in an anatomical joint of a patient, comprising:
an implant selected from a predefined set of implants having varying dimensions;
an insert tool corresponding to the selected implant, selected from a predefined set of insert tools, each having an implant engaging portion that has a surface curvature that substantially corresponds to the surface curvature of the articulating surface of an implant in the predefined set of implants, wherein the insert tool is configured to be used for pressing said implant to a suitably shaped implant receiving surface that has been e.g. drilled, milled, and/or sawed into the joint at the site of diseased cartilage and/or bone; and
a guide tool comprising a contact surface configured to have a shape and contour that is designed to correspond to and to fit the contour of the cartilage and/or the bone in a predetermined area that is located to allow the guide tool to guide a tool for creating an implant receiving surface that is shaped for receiving said selected implant at a site of diseased cartilage and/or bone.

11. Surgical kit according to claim 10, further comprising an implant dummy corresponding to the selected implant, selected from a predefined set of implant dummies having dimensions corresponding to the predefined set of implants.

12. Method for inserting an implant comprising a positioning mark at a site of determined damage in an anatomical joint of a patient, for repairing damage in the anatomical joint, the method comprising:
attaching a guide tool to an articulating surface in the joint, the guide tool comprising a contact surface configured to have a shape and contour that is designed to correspond to and to fit the contour of the cartilage and/or the bone in a predetermined area that is located to allow the guide tool to guide a tool for creating an implant receiving surface that is shaped for receiving said implant at a site of diseased cartilage and/or bone;
creating an implant receiving surface at the site of diseased cartilage and/or bone, e.g. by drilling, milling, and/or sawing the implant receiving surface, using the guide tool;
removing the guide tool from the anatomical joint;
rotating the implant so that the positioning mark on the implant is aligned with the marking on the cartilage;
placing the implant on the implant receiving surface; and
pressing the implant to the implant receiving surface using an insert tool.

13. Method according to claim 12, further comprising making a marking on the cartilage at the side of the implant receiving surface, before removing the guide tool from the anatomical joint.

14. Method according to claim 12, further comprising verifying that the implant receiving surface fits the implant, using an implant dummy.

15. Method according to claim 12, further comprising applying an adhesive on the implant receiving surface, and/or on a bone contacting surface of the implant, before placing the implant on the implant receiving surface.

16. Method according to claim 12, wherein the implant receiving surface comprises a recess, into which at least a part of the implant is inserted.

17. Method according to claim 12, adapted to be at least partially automatically performed by a robot.
